(19) United States
(12) Patent Application Publication
(10) Pub. No.: US 2017/0239187 A1
(43) Pub. Date: Aug. 24, 2017

(54) STABLE DOSAGE FORM ARTICLES FOR ORAL ADMINISTRATION

(71) Applicant: Capsugel Belgium NV, Bornem (BE)

(72) Inventors: Celine Casi, Brunstatt (FR); Jan Emiel Godelieve Vertommen, Grimbergen (BE)

(73) Assignee: Capsugel Belgium NV, Bornem (BE)

(21) Appl. No.: 15/438,565

(22) Filed: Feb. 21, 2017

Related U.S. Application Data
(60) Provisional application No. 62/298,040, filed on Feb. 22, 2016.

(51) Int. Cl. A61K 9/48; A61K 31/7076

(52) U.S. Cl. CPC A61K 9/4816; A61K 31/7076

(57) ABSTRACT

Novel delivery systems for highly hygroscopic active materials is disclosed. The delivery system comprising a hard capsule filled with a liquid composition consisting of a hygroscopic active material dispersed in a non-aqueous carrier. The carrier comprising, or consisting of, one or more lipophilic lipid substances and one or more rheology modifiers.

### CROSS REFERENCE TO RELATED APPLICATION

This application claims the benefit of U.S. Provisional Application No. 62/298,040, filed Feb. 22, 2016, which is incorporated by reference herein.

### FIELD

The present disclosure relates to dosage form delivery systems and methods of making and using the same.

### BACKGROUND

Capsules are widely used in the pharmaceutical field as oral dosage form containers for administration to humans and animals of, e.g., pharmaceuticals, veterinary products, foods, and dietary supplements.

The traditional material for forming the capsule shell is gelatin. Conventional hard capsules are made with gelatin by dip molding processes. For the industrial manufacture of pharmaceutical capsules gelatin is preferred for its gelling, film forming and surface active properties. Traditionally, gelatin is produced by extraction from collagen containing mammalian tissues, particularly such as pig skin and bovine bone.

Gelatin has some disadvantages, however, which make it necessary to have other capsule shell materials available. A major unfavorable aspect is the animal origin of gelatin. Other disadvantages are the inconveniences of relatively high water content (10-17%) and the loss of elasticity with decreasing water content. Furthermore gelatin capsules are sensitive to heat and humidity which affect the usability of the product. In particular soft gelatin capsules are known to aggregate under hot and humid conditions. Under dry conditions gelatin films may induce static charge build up affecting later processing. It has long been recognized that gelatin capsules become brittle when they lose some of their moisture content. Water acts as a plasticizer in gelatin films and when the level falls below about 10%, they become very brittle. On the other hand when the water content increases the capsule shell can weaken and finally leakage will occur.

The formulation of drugs into soft gelatin capsules has gained popularity throughout the past decade due to the many advantages of this dosage form. A disadvantage of soft gelatin capsules is that the atmospheric moisture may permeate into the capsule shell or into the fill. Volatile components in soft gelatin capsules may escape into the atmosphere. These liquid mixtures are very often dispersions which show a reduced bioavailability in particular in the gastrointestinal tract. In order to overcome these problems it has been suggested to use more complex solvent systems in gelatin capsules.

Other disadvantages are the problems related to capsule shell/contents interactions and/or unacceptable taste and smell.

### SUMMARY

The instant disclosure relates to an innovative chemically stable dosage form. Certain embodiments of the present disclosure provide a novel delivery system that overcomes the problems of the state of the art whilst at the same time limiting complexity and cost of production without impacting efficacy.

In a first aspect, the disclosure relates to a comestible dosage form article for oral administration comprising a liquid fill composition, said liquid film composition comprising a dispersion of a hygroscopic active material in a carrier, wherein the carrier consists of a non-aqueous composition comprising one or more lipophilic lipid substances and one or more rheology modifiers.

In a second aspect, the disclosure relates to a method of making such dosage forms.

In a third aspect, the disclosure relates to the use of a non-aqueous liquid carrier consisting of a lipophilic lipid substance and a rheology modifier to stabilize a hygroscopic active material contained in a hard capsule.

Surprisingly, the dosage form according to the present disclosure confers high chemical stability over time.

### DETAILED DESCRIPTION

Advantages of capsules over other dosage forms may include better patient compliance, greater flexibility in dosage form design, and less expensive manufacturing processes. Pharmaceutical capsules are conventionally divided into soft shell capsules (hereinafter softgel capsules) and hard shell capsules (hereinafter hard capsules).

Hard capsule shells are generally manufactured using dip molding processes involving the use of pins dipped into solutions of the different ingredients that are needed for the making of the capsule shell containers. Methods for the manufacturing of soft gelatin or softgel capsule shells are also known in the art and are different from hard capsule shell manufacturing. Manufacturing of soft gelatin or softgel capsule shells at a production scale was introduced by Robert Pauli Scherer in 1933 with the invention of a rotary die encapsulation machine. The rotary die process involves continuous formation of a heat seal between two ribbons of gelatin simultaneous with dosing of the fill liquid into each capsule. Although manufacturing process speed and efficiency has improved with time, the basic manufacturing principle remains essentially unchanged. Before the encapsulation process takes place, two sub-processes are often carried out simultaneously, yielding the two components of a softgel capsule: (a) the gel mass which will provide the softgel capsule shell, and (b) the fill matrix for the softgel capsule contents. Softgel capsules have a continuous gelatin shell surrounding a liquid core, and are formed, filled, and sealed in one operation.

Softgel capsule walls are typically thicker than two-piece hard gelatin capsules, contain a higher level of residual water in the walls, and their walls comprise plasticizers such as, for example, glycerol, sorbitol and/or propylene glycol to make the shell elastic. Processes for making softgel capsule shells are known, and softgel capsules are available commercially. See, e.g., Aulton, M., Aulton's Pharmaceutics: The Design & Manufacture of Medicines, 527-533 (Kevin M G Taylor, Ed., 3rd Ed., 2001).

The bioavailability of hydrophobic drugs can be significantly increased when formulated into soft gelatin capsules. Many problems associated with tableting, including poor compaction and lack of content or weight uniformity, can be eliminated when a drug is incorporated into such dosage forms. Soft gelatin capsules generally contain the medicament dissolved or dispersed in oils or hydrophilic liquids (i.e., fill liquid). The inherent flexibility of the soft gelatin capsule is due to the presence of plasticizers and residual moisture in the capsule shell.

The above-noted disadvantages are addressed by the technology described herein. Certain embodiments of the present disclosure relate to delivery systems comprising a hard capsule filled with a liquid composition consisting of a hygroscopic active material dispersed in a non-aqueous carrier. The carrier comprising, or consisting of, one or more lipophilic lipid substances and one or more rheology modifiers. The liquid fill (the active material and the carrier considered together) may be a dispersion. The disclosure further discloses methods of making and the use thereof.

The terms “chemical stability or “chemically stable” as used herein mean the amount of SAMe presents in each formulation after 3 months of storage under room conditions (temperature comprised between 18 and 22° C. and relative humidity comprised between 40 and 60% RH). Various embodiments will now be described to provide an overall understanding of the principles of the structure, function, manufacture, and use of dosage form articles, methods and uses disclosed herein. Those of ordinary skill in the art will immediately understand that features described in connection with one example embodiment can be combined with the features of other example embodiments without generalization from the present disclosure.

In an embodiment, the disclosure contemplates a non-gelatin comprising hard capsule for oral administration and for containing therein a liquid fill composition, the fill composition comprising, or consisting of, one or more hygroscopic active materials (particularly sensitive to humidity and/or temperature), and a non-aqueous carrier comprising, or consisting of, a lipophilic lipid substance and a rheology modifier.

In an embodiment, the hard capsule is a dip-molded capsule obtained from a film forming composition comprising at least one polysaccharide or polysaccharide derivative. The polysaccharide or derivative thereof may be cellulose, cellulose derivatives, starch, modified starches, pullulan, dextran or the like and mixtures of any of the foregoing.

In an embodiment, the hard capsule is made of a material selected from hydroxypropylmethylcellulose (HPMC), pullulan, and mixtures thereof. Suitable commercially available hard capsules include: Vcaps®, Vcaps®Plus, DRcaps®, and Plantcaps® manufactured and sold by Capsugel®. Advantageously, such non-gelatin hard capsules have a low moisture content (or loss on drying, LOD) of typically less than 6.5% w/w.

In an embodiment, the hard capsule herein has a loss on drying (LOD), after 10 days of storage at 25° C. and at relative humidity of 53% (according to the test method described herein) of 6.5%, preferably 6%, more preferably 5.5%, more preferably 5%, even more preferably 4.5%, even more preferably 4%, even more preferably 3.5%, even more preferably 3%, even more preferably 2.5%, by weight or less. An advantage of this embodiment is to ensure the capsule does not act as a moisture sink and moisture migration from the capsule wall into the liquid fill (or vice-versa) is limited. Moreover, the liquid fill described herein does not migrate into the capsule wall or interact with it.

Suitable cellulose derivatives are selected from alkylcelluloses, hydroxyalkylcelluloses, hydroxyalkylalkylcelluloses, carboxyalkylcelluloses, and carboxyalkyl-alkylcelluloses including, but not limited to, members selected from methyl cellulose, ethyl cellulose, hydroxymethylcellulose, hydroxyethyl cellulose, hydroxyethylethyl cellulose, hydroxypropyl cellulose, hydroxypropylmethyl cellulose (HPMC), hydroxyethylmethyl cellulose, hydroxybutylmethylcellulose, cellulose acetylphtalate (CAP), sodium carboxymethyl cellulose and mixtures of any of the foregoing. Especially preferred is hydroxpropylmethyl cellulose (H PMC).

Additional cellulose derivatives include cellulose ethers. Suitable cellulose ethers are selected from alkyl- and/or hydroxyalkyl substituted cellulose ether with 1 to 4 carbon atoms in the alkyl chains, and are preferably selected from methyl cellulose ether, hydroxyethyl cellulose ether, hydroxypropyl cellulose ether, hydroxyethylmethyl cellulose ether, hydroxyethylethyl cellulose ether, hydroxypropylmethyl cellulose ether or the like and mixtures of any of the foregoing. Especially preferred is hydroxpropylmethyl cellulose ether.

A particular group of cellulose derivatives are those selected from methyl cellulose, ethyl cellulose, hydroxymethyl cellulose, hydroxyethyl cellulose, hydroxyethylethyl cellulose, hydroxypropyl cellulose, hydroxypropylmethyl cellulose (HPMC), hydroxyethylmethyl cellulose, hydroxybutylmethyl cellulose, cellulose acetylphthalate (CAP), sodium carboxymethyl cellulose, methyl cellulose ether, hydroxyethyl cellulose ether, hydroxypropyl cellulose ether, hydroxyethylmethyl cellulose ether, hydroxyethylethyl cellulose ether, and hydroxypropylmethyl cellulose ether.

Beside starch, modified starches can be used, such as starch ethers and oxidized starches, carboxymethyl starches, hydroxyalkylated starches, and succinated starches, more particularly hydroxypropylated starch (HPS) or hydroxyethylated starch (HES) or mixtures thereof can be used as a film forming material in the film forming composition for obtaining the capsules according to the present disclosure. Modified starches disclosed in U.S. Pat. No. 6,635,275 B1 are suitable for the present disclosure. Most preferred modified starch is HPS.

In one particular embodiment the capsule is a formed body obtained from a film forming composition comprising at least one polysaccharide or polysaccharide derivative or mixture thereof from about 90% to 99% by weight of the final capsule. The viscosity of the polysaccharide, polysaccharide derivative or mixture thereof is from 3 to 15 cps in 2% aqueous solution at 20° C., preferably from 5 to 10 cps, and more preferably 6 cps.

In one particular embodiment the capsule is a formed body obtained from a film forming composition comprising at least one polysaccharide or polysaccharide derivative or mixture thereof from about 5% to about 40% of the aqueous solution. Most preferred the capsule is a formed body obtained from about 17% of the aqueous solution.

In a preferred embodiment according to the present disclosure the capsule is a hard HPMC capsule. The capsule may be a two-piece hard capsule, which can be produced by dipping method or produced by injection-moulding method. The capsule can be a one compartment dosage form as well as a multiple compartment dosage form. Suitable methods for making such two-piece capsules include those methods found in the art. For example, a two-piece capsule may be made by the dipping method described in “Pharmaceutical Capsules” second edition, from Fridrun Podczeck and Brian E Jones et al, page 80-84.

The film forming composition for obtaining the capsule of the present disclosure may optionally comprise a suitable gelling agent selected from known gelling agents with the proviso that such gelling agent improves the gelling capability of the capsule and does not interact with the liquid fill comprising the carrier composition and/or the active agent(s). Examples of suitable gelling agents include alginic acid, sodium alginate, potassium alginate, calcium alginate, agar, carrageenan, carob gum, and gellan gum. Most preferred gelling agent is gellan gum or carrageenan.

The capsule may optionally be coated with a suitable coating agent such as a those selected from cellulose acetate phthalate, polyvinyl acetate phthalate, methacrylic acid gelatines, hypromellose phthalate, hydroxypropylmethyl cellulose phthalate, hydroxyalkyl methyl cellulose phthalates, hydroxypropyl methylcellulose acetate succinate and mixtures thereof to provide e.g., enteric properties.

In a further embodiment of the disclosure, the capsules can additionally be sealed using means known in the art such as banding or liquid sealing technology. Examples of suitable sealing techniques are those described in WO 01/08631, U.S. Pat. No. 2,962,851, and in “Pharmaceutical capsules”, second edition, from Fridrun Podczeck and Brian E Jones et al, pages 182-184. More preferably the capsules are sealed using a hydro alcoholic solution for example, as described in WO 01/08631.

The liquid fill according to the present disclosure consists of a dispersion of a hygroscopic active material in a non-aqueous carrier. The non-aqueous carrier comprising, or consisting of a lipophilic lipid substance and a rheology modifier.

Hygroscopic active materials for use herein may be selected from SAMe, choline chloride, chondroitine sulfate, collagen, L-Carnitine L-tartrate, L-Arginine Ethylester Dihydrochloride

In particular, S-adenosyl L-methionine (SAMe) is a particularly difficult material to encapsulate using state of the art delivery systems and processes. Without being limited to a particular theory, it is currently believed that this is particularly due to the hygroscopic nature and low pH of SAMe, which does not permit easy encapsulation since the initial water content of the capsule shell (and/or water content in the carrier system) has an adverse effect on the compound. Moreover, if enteric coated, the coating has to be optimized for the desired availability of SAMe at the target site e.g., anterior part of the intestine.

S-adenosylmethionine participates in a great number of metabolic processes of fundamental importance for human organism, and consequently its deficiency lies at the basis of many organic malfunctions. S-adenosylmethionine is a natural molecule synthesized from the amino acid methionine in the presence of magnesium and adenosine triphosphate (ATP). The SAMe molecule is a carrier of methyl groups and provides a sulfur molecule as well. The liver is a site of methylation and sulfation reactions necessary for detoxification, and can use SAMe to assist in these processes. SAMe is also a cofactor in several metabolic reactions. By donating its methyl group, SAMe is converted to adenosylhomocysteine which, in turn, is rapidly hydrolysed to adenosine and homocysteine and eventually to the amino acid, cysteine. S-adenosylmethionine is necessary for the production of Glutathione, the primary antioxidant found in the liver. SAMe has also been found effective in the treatment of Cholestasis. SAMe is also used in the treatment of fibromyalgia, osteoarthritis and depression. It is therefore highly desirable to provide an effective and stable delivery system for such active.

In a preferred embodiment, the hygroscopic active material consists of S-adenosyl L-methionine (SAMe). The combination of particular carrier and capsule described herein has been found to be a particularly suitable delivery system for such material.

Suitable lipophilic lipid substances may be selected from mineral oil; light mineral oil; natural oils such as vegetable, corn, canola, sunflower, soybean, olive, coconut, cocoa, peanut, almond, cottonseed, persic, rapeseed, sesame, squalane, castor, cod liver, fish, oils; hydrogenated vegetable oil; partially hydrogenated oils; beeswax; polyethoxylate beeswax; paraffin; normal waxes; medium chain monoglycerides, diglycerides and triglycerides, higher aliphatic alcohols, higher aliphaticacids; long chain fatty acids; saturated or unsaturated fatty acids; hydrogenated fatty acids; fatty acid glycerides; polyoxyethylated oleic glycerides; monoglycerides and diglicerides; mono-, bi- or tri-substituted glycerides; glycerol mono-oleate esters; glycerol mono-caprate; glyceryl monocaprylate; mono and diglycerides of fatty acids, glyceryl monostearate, propylene glycol dicaprylate; propylene glycol monolaurate; glyceryl palmitostearate; glyceryl behenate; diethyleneglycol; palmitostearate; polyethyleneglycol stearate; polyoxyethyleneglycol palmitostearate; glyceryl mono palmitostearate; cetyl palmitate; polyethyleneglycol palmitostearate; dimethylpolysiloxane; mono- or di-glyceryl behenate; and mixtures thereof.

The most preferred lipophilic lipid substance comprises, preferably consists of, soybean oil.

Suitable rheology modifiers may be selected from hydrogenated vegetable oil, partially vegetable oil, beeswax, mono and diglycerides of fatty acids, glyceryl palmitostearate, glyceryl behenate and glyceryl monostearate

The most preferred rheology modifier comprises, preferably consists of, glyceryl monostearate.

The hygroscopic active material is dispersed (rather than solubilized) in the non-aqueous carrier described herein. An advantage of particularly forming a dispersion is that the hygroscopic material becomes completely insulated by the lipophilic lipid substance without the need for complex processing like individual coating of the active material particles, such resulting in a fairly quick and inexpensive process for limiting any further humidity interaction between the environment and the active.

However, not adding water in the carrier (and thus not promoting solubilization) typically results in low amounts of active that could be added to the carrier. To the contrary, it is desirable to introduce high solids content of active within the liquid fill.

It has been advantageously found that with non-aqueous carriers described herein a high solids content of active may be attained.

In an embodiment, the dispersion of active material in the liquid carrier has a solids content of at least about 35%, preferably at least about 40%, more preferably from about 45% to about 55%, by weight of the liquid fill composition.

The addition of one or more rheology modifiers as described herein has the advantage of maintaining the active particles dispersed within the carrier for longer periods of time during storage (low shear) thus resulting in reduced separation/deposition and thus better physical stability over time by ensuring proper insulation from humidity.

In an embodiment, the weight ratio of the lipophilic lipid substance to rheology modifier is from about 3.0 to about 12.0, preferably from about 5.0 to about 10.0, more preferably from about 7.0 to about 9.0.

In an embodiment, the weight ratio of the hygroscopic active material to the lipophilic lipid substance is from about 0.8 to about 1.5, preferably from about 0.9 to about 1.2.

In an embodiment, the rheology modifier is present in amount of from about 0.1% to about 12.0%, preferably from about 1% to about 10.0%, more preferably from about 3% to about 7%, by weight of the total liquid fill composition.

The carrier described herein achieves a balance between ensuring a sufficiently low viscosity is attained for filling (at higher shear rates) whilst at the same time ensuring a sufficiently high viscosity (at lower shear rates) for maintaining the high solids content of active material well dispersed within the carrier. Without wishing to be bound by theory, it is believed that a carrier in the proportions described herein allows for a sufficiently high yield stress to be attained in order to achieve the desired long-term well dispersed behavior.

In an embodiment, a method is provided for making dosage forms described herein, the method comprising the steps of: providing a hard capsule as described herein; filling the hard capsule with a liquid fill composition as described herein; and sealing and/or banding the hard capsule such that the liquid fill composition is completely enclosed within said hard capsule.

The compositions and methods of the present disclosure are useful for, but not limited to, for example, oral administration to humans or animals.

Loss on drying (LOD) test method:

The hard capsule for use herein is characterized in that its capsule film has a low equilibrium moisture content. The equilibrium moisture of the capsule film can be evaluated from the moisture content of the film when a hard capsule is placed under a specific relative humidity condition. In particular, the hard capsule herein exhibits a loss on drying after 10 days of storage at 25° C. and at a relative humidity of 53% of less than 6.5% by weight. This is preferably 5.8% by weight or less, more preferably 5.5% by weight or less, and still more preferably 5% by weight or less.

The “loss on drying (LOD)” as used herein means a decreased moisture content upon heating and drying a capsule film at about 105° C. for 8 hours. The loss on drying after 10 days of storage at 25° C. and at a relative humidity of 53% can be measured by the method described below.

Measurement method for loss on drying a sample (hard capsule) weighing 0.5 to 5.0 g is placed into a desiccator having an atmosphere in which the humidity is made constant by including a saturated aqueous solution of magnesium nitrate inside the desiccator, and then the desiccator is sealed and stored at 25° C. for 10 days. In the presence of a saturated aqueous solution of magnesium nitrate, the relative humidity can be adjusted to approximately 53%. The weight (wet weight) of the sample after storage is measured, and the sample is then heated at about 105° C. for 8 hours. Then, the weight (dry weight) of the sample is measured again. From the difference in the weight of the sample between before drying (wet weight) and after drying (dry weight), the amount of moisture decrease (loss on drying) upon heating and drying at about 105° C. for 8 hours is calculated according to the following equation:

Loss on drying (% by weight)=[(Wet weight of capsule−Dry weight of capsule)/Wet weight of capsule]×100

As for the hard capsule herein, the loss on drying after 10 days of storage at 25° C. and at a relative humidity of 12%, 22%, 33%, or 43% is 1.1% by weight or less, 2.1% by weight or less, 3.2% by weight or less, and 4.7% by weight or less, respectively. The hard capsule herein does not need to satisfy all of the loss-on-drying conditions at the above-mentioned relative humidity levels. Satisfying at least one of the conditions is sufficient. The hard capsule herein will preferably satisfy two or more conditions, more preferably three or more conditions, and still more preferably all four conditions.

The relative humidity conditions can each be attained using, in place of the saturated aqueous solution of magnesium nitrate, a saturated aqueous solution of lithium chloride, potassium acetate, magnesium chloride, or potassium carbonate in the above-mentioned method. More specifically, in the presence of the saturated aqueous solution of lithium chloride, potassium acetate, magnesium chloride, or potassium carbonate, the relative humidity can be set to approximately 12%, approximately 22%, approximately 33%, or approximately 43%, respectively. The loss on drying after 10 days of storage at 25° C. and at a relative humidity of 12% is preferably 1% by weight or less, and more preferably 0.9% by weight or less; the loss on drying after 10 days of storage at 25° C. and at a relative humidity of 22% is preferably 1.9% by weight or less, and more preferably 1.6% by weight or less; the loss on drying after 10 days of storage at 25° C. and at a relative humidity of 33% is preferably 2.8% by weight or less, and more preferably 2.4% by weight or less; and the loss on drying after 10 days of storage at 25° C. and at a relative humidity of 43% is preferably 4.2% by weight or less, and more preferably 3.6% by weight or less. Further, the hard capsule herein is characterized in that its capsule film has a low hygroscopicity.

The hygroscopicity of the capsule film can be evaluated from the relationship between relative humidity and the loss on drying (capsule moisture value (%)) of the capsule film at that relative humidity, as shown below.

The moisture content of a sample (hard capsule) weighing 0.5 to 5.0 g is reduced with a silica gel, and the obtained sample is then placed into a desiccator, the desiccator having an atmosphere in which the humidity is made constant by including a saturated aqueous solution of lithium chloride, potassium acetate, magnesium chloride, potassium carbonate, magnesium nitrate, sodium chloride, or monobasic potassium phosphate inside the desiccator. Thereafter, the desiccator is sealed, and the sample is stored at 25° C. as is. In the presence of the saturated aqueous solution of lithium chloride, potassium acetate, magnesium chloride, potassium carbonate, magnesium nitrate, sodium chloride, or monobasic potassium phosphate, the relative humidity can be set to approximately 12%, approximately 22%, approximately 33%, approximately 43%, approximately 53%, approximately 75%, or approximately 96%, respectively.

The weight (wet weight) of the sample after storage is measured, and the sample is then heated at about 105° C. for 8 hours. Subsequently, the weight (dry weight) of the sample is measured again. From the difference in the weight of the sample before drying (wet weight) and after drying (dry weight), the amount of moisture loss (loss on drying) upon heating and drying at about 105° C. for 8 hours is calculated according to the following equation:

Capsule moisture value (%)=[(wet weight of capsule at a relative humidity−dry weight of capsule)/wet weight of capsule at a relative humidity]×100

Subsequently, the ratio (%) of the moisture value (%) of the capsule at a specific relative humidity (%) to the relative humidity (%) is calculated, and the hygroscopicity (%) of the capsule film is evaluated from this value:

Moisture absorption properties (%)=(capsule moisture value/the relative humidity)×100

The hygroscopicity (%) at 25° C. and at a relative humidity of 12% is preferably 8.3% or less and more preferably 7.5% or less. The hygroscopicity (%) at 25° C. and at a relative humidity of 22% is preferably 8.6% or less and more preferably 7.3% or less. The hygroscopicity (%) at 25° C. and at a relative humidity of 33% is preferably 8.5% or less and more preferably 7.3% or less. The hygroscopicity (%) at 25° C. and at a relative humidity of 43% is preferably 9.8% or less and more preferably 8.4% or less. The hygroscopicity (%) at 25° C. and at a relative humidity of 53% is preferably 10.4% or less and more preferably 9.4% or less.

Chemical Analytical Tests Method:

Level of SAMe is measured by HPLC method. First step is the preparation of the sample: sample is solubilized in HCl 0.1 N in order to solubilize SAMe. This solution is injected on HPLC Hitachi LaChrom L7000. Chromatogram obtained are analyzed in order to obtain value of SAMe presents in the sample.

### Examples

Table 1 gives examples of the composition in weight percentages of formulation (by weight of fill).

TABLE 1

| | Formula A | Formula B | Formula C | Formula D | Formula E | Formula F |
|---|---|---|---|---|---|---|
| Raw material | (% w/w) | (% w/w) | (% w/w) | (% w/w) | (% w/w) | (% w/w) |
| SAMe¹ | — | 40 | — | — | — | 40 |
| SAMe² | 35 | — | 48 | 40 | 35 | — |
| Glycerol³ | 65 | 57 | — | — | — | — |
| Water demineralized | — | 3 | — | — | — | — |
| Soybean oil⁴ | — | — | 46 | 50 | 55 | 50 |
| Glycerol monostearate⁵ | — | — | 6 | 10 | 10 | 10 |
| Total | 100 | 100 | 100 | 100 | 100 | 100 |

¹S-Adenosyl-L-methionine PATES from Gnosis
²SAM-e PATES dried mix from Gnosis, reference 0-S28
³Glycerol from Cooper
⁴Soybean oil from Olvea
⁵Glycerol monostearate from Oleon

Preparation of the Dosage from:

For formulation based on lipophilic lipid excipients preparation protocol is the following: Oil and rheology modifier are weighed and mixed together. Formulation is heated at appropriate temperature to solubilize rheology modifier. Formulation is cool down to an adapted temperature to add SAM-e previously weighed. SAM-e is dispersed in the formulation. Final formulation is filled in empty capsules at appropriate filling temperature depending on rheological characteristics of the formulations. Capsules are sealed or banded.

For formulation based on aqueous carriers preparation protocol is the following: aqueous carriers are weighed and mixed together. SAM-e previously weighed is added and formulation is mixed until complete solubilization of the SAM-e (formulation could be heated if necessary). Final formulation is filled in empty capsules at appropriate filling temperature depending on rheological characteristics of the formulations. Capsules are sealed or banded.

Table 2—is a table of comparative data of chemical stability of capsules filled with S-Adenosyl methionine formulations (of table 1) after 3 months of storage under room conditions.

| | Formula B | Formula C |
|---|---|---|
| Amount of SAM-e measured after | 183 mg/capsule | 373 mg/capsule |
| 3 months under room conditions | | |

Every document cited herein, including any cross referenced or related patent or application, is hereby incorporated herein by reference in its entirety unless expressly excluded or otherwise limited. The citation of any document is not an admission that it is prior art with respect to any embodiment disclosed or claimed herein or that it alone, or in any combination with any other reference or references, teaches, suggests or discloses any such embodiment. Further, to the extent that any meaning or definition of a term in this document conflicts with any meaning or definition of the same term in a document incorporated by reference, the meaning or definition assigned to that term in this document shall govern.

While particular embodiments of the present disclosure have been described, it would be obvious to those skilled in the art that various other changes and modifications can be made without departing from the spirit and scope of the disclosure. It is therefore intended to cover in the appended claims all such changes and modifications that are within the scope of this disclosure.

We claim:

1. A comestible dosage form article for oral administration, comprising a liquid fill composition, which comprises a dispersion of a hygroscopic active material in a non-aqueous carrier composition comprising a lipophilic lipid substance and a rheology modifier.

2. The dosage form article according to claim 1, wherein the dosage form article is a non-gelatin hard capsule having a loss on drying (LOD) of 6.5% by weight or less, after 10 days of storage at 25° C. and at relative humidity of 53%.

3. The dosage form article according to claim 2, wherein the non-gelatin hard capsule comprises a material selected from hydroxypropyl methylcellulose (HPMC), pullulan, and mixtures thereof.

4. The dosage form article according to claim 1, wherein the hygroscopic active material consists of S-adenosyl L-methionine (SAM-e), choline chloride, chondroitine sulfate, collagen, L-Carnitine L-tartrate, L-Arginine Ethylester Dihydrochloride, or salts thereof.

5. The dosage form article according to claim 1, wherein the lipophilic lipid substance is selected from mineral oil; light mineral oil; natural oils selected from vegetable, corn, canola, sunflower, soybean, olive, coconut, cocoa, peanut, almond, cottonseed, persic, rapeseed, sesame, squalane, castor, cod liver, and fish oils; hydrogenated vegetable oil; partially hydrogenated oils; beeswax; polyethoxylate beeswax; paraffin; normal waxes; medium chain monoglycerides, diglycerides and triglycerides, higher aliphatic alcohols, higher aliphaticacids; long chain fatty acids; saturated or unsaturated fatty acids; hydrogenated fatty acids; fatty acid glycerides; polyoxyethylated oleic glycerides; monoglycerides and diglicerides; mono-, bi- or tri-substituted glycerides; glycerol mono-oleate esters; glycerol mono-caprate; glyceryl monocaprylate; mono and diglycerides of fatty acids, glyceryl monostearate, propylene glycol dicaprylate; propylene glycol monolaurate; glyceryl palmitostearate; glyceryl behenate; diethyleneglycol; palmitostearate; polyethyleneglycol stearate; polyoxyethyleneglycol palmitostearate; glyceryl mono palmitostearate; cetyl palmitate; polyethyleneglycol palmitostearate; dimethylpolysiloxane; mono- or di-glyceryl behenate; and mixtures thereof.

6. The dosage form article according to claim 1, wherein the lipophilic lipid substance comprises soybean oil.

7. The dosage form article according to claim 1, wherein the rheology modifier is selected from hydrogenated vegetable oil, partially vegetable oil, beeswax, mono and diglycerides of fatty acids, glyceryl palmitostearate, glyceryl behenate, glyceryl monostearate, and mixtures thereof.

8. The dosage form article according to claim 1, wherein the rheology modifier comprises glycerol monostearate.

9. The dosage form article according to claim 1, wherein the lipophilic lipid substance consists of soybean oil and the rheology modifier consists of glycerol monostearate.

10. The dosage form article according to claim 1, wherein the carrier consists of the lipophilic lipid substance and the rheology modifier.

11. The dosage form article according to claim 1, wherein the lipophilic lipid substance and the rheology modifier have a weight ratio of the lipophilic lipid substance to rheology modifier of from about 3.0 to about 12.0.

12. The dosage form article according to claim 1, wherein the lipophilic lipid substance and the rheology modifier have a weight ratio of the lipophilic lipid substance to rheology modifier of from about 7.0 to about 9.0.

13. The dosage form article according to claim 1, wherein the hygroscopic active material and the lipophilic lipid substance have a weight ratio of the hygroscopic active material to the lipophilic lipid substance of from about 0.8 to about 1.5.

14. The dosage form article according to claim 1, wherein the dispersion of hygroscopic active material in the liquid carrier has a solids content of at least about 35% to about 55% by weight of the liquid fill composition.

15. The dosage form article according to claim 1, wherein the dispersion of hygroscopic active material in the liquid carrier has a solids content of from about 45% to about 55% by weight of the liquid fill composition.

16. The dosage form article according to claim 1, wherein the rheology modifier is present in amount of from about 0.1% to about 12.0% by weight of the total liquid fill composition.

17. The dosage form article according to claim 1, wherein the rheology modifier is present in amount of from about 3.0% to about 7.0% by weight of the total liquid fill composition.

18. A method of making a dosage form article, comprising:
providing a hard capsule;
filling the hard capsule with a liquid fill composition comprising a dispersion of a hygroscopic active material in a carrier, wherein the carrier consists of a non-aqueous composition comprising one or more lipophilic lipid substances and one or more rheology modifiers; and
sealing and/or banding the hard capsule such that the liquid fill composition is completely enclosed within said hard capsule.

19. A method, comprising, stabilizing a hygroscopic S-adenosyl L-methionine (SAMe) active material by combining the hygroscopic S-adenosyl L-methionine (SAMe) active material with a non-aqueous carrier consisting of a lipophilic lipid substance and a rheology modifier.

20. The method according to claim 19, wherein the lipophilic lipid substance is soybean oil and the rheology modifier is glycerol monostearate.
